SEQUENCE LISTING (6 sequences)

SEQ ID NO: 1 - DNA, 26 bp, Artificial Sequence
  oligonucleotide
tggatggaattcagtttctcgcatca

SEQ ID NO: 2 - DNA, 23 bp, Artificial Sequence
  oligonucleotide
gcttcaatcaaaagtggtttcaa

SEQ ID NO: 3 - DNA, 38 bp, Artificial Sequence
  oligonucleotide
aggaaaaaagcggccgcgccaccatgcgaccccagggc

SEQ ID NO: 4 - DNA, 25 bp, Artificial Sequence
  oligonucleotide
ccgctcgagattttggtagttcttc

SEQ ID NO: 5 - DNA, 1236 bp, Homo sapiens <s300> gi|34147546|ref|NM_138455.2
ctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaaaaaaaaaaaaaaaaa

SEQ ID NO: 6 - protein, 243 aa, Homo sapiens <s300> gi|19923989|ref|NP_612464.1
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

(19) United States
(12) Patent Application Publication
(10) Pub. No.: US 2010/0183512 A1
(43) Pub. Date: Jul. 22, 2010

(54) USE OF CTHRC1 IN DIAGNOSING CANCER OF LIVER

(76) Inventors: Wenxin QIN, Shanghai (CN); Genfu Yao, Shanghai (CN); Xiaozhen Wan, Shanghai (CN); Shengli Yang, Shanghai (CN); Jianren Gu, Shanghai (CN)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/614,762

(22) Filed: Nov. 9, 2009

Related U.S. Application Data
(63) Continuation of application No. PCT/CN2007/070006, filed on May 9, 2007.

(51) Int. Cl. A61K 51/06; C07H 21/04; C07K 14/47; C07K 16/30; C12Q 1/68

(52) U.S. Cl. 424/1.73; 536/23.5; 530/395; 530/389.7; 435/6

(57) ABSTRACT

Use of protein CTHRC1 or its nucleic acid in preparation for reagents or kits for diagnosing cancer of liver, the method for diagnosing cancer of liver by using the nucleic acid of CTHRC1, kit comprising the antibody to CTHRC1 or nucleic acid probe specific for the CTHRC1 protein and label, and the method for detecting the specific expression of protein CTHRC1 are disclosed.

### TECHNICAL FIELD

The present invention relates to molecular biology, especially genetic diagnosis. Specifically, the present invention relates to the use of CTHRC-1 in diagnosis of liver cancer.

### BACKGROUND ART

Hepatocellular carcinoma (HCC) is one of the malignant tumor with high incidence and mortality in the world. Each year, about 50% HCC onset of the world was taken place in China [1]. The development and metastasis of HCC is a complicated network regulation system involving multiple genes and signaling pathways.

Mammalian CTHRC1 (Collagen triple helix containing 1) was first discovered in the screen of differentially expressed sequences of normal rat artery and globular injured artery, which included one N-terminal signal peptide, collagen triple helix of 36 aa and C-terminal globular domain[2]. The amino acid sequences of the said gene are 92% homologous between human and rat[3]. However, it was not seen to have any specific link to human hepatoma cells.

CTHRC1 gene is transiently expressed at injured site of rat artery, in fibroblasts in artery outer membrane remodeling, and in the smooth muscle cells in newly generated inner membrane. In rat fibroblasts, high expression of the said gene will cause cell migration and inhibition of the synthesis of type I collagen [2], which indicates that CTHRC1 involves the damage repair of blood vessel by inhibiting deposition of collagen matrix and promoting cell migration. More and more evidences show that the tissue reparation is closely linked to tumorigenesis [4-6].

The relationship between CTHRC1 gene and human tumor was first reported in breast cancer. It was found from cDNA chips and in situ hybridization that stroma cells in breast cancer expressed the mRNA of the said gene [7,8]. Later there were researches reporting that the said gene abnormally expressed in melanoma, and involved invasion and migration of cancer cells [3]. Many reports proved that tumor microenvironment was important for the growth, invasion and migration of tumor cells [9-11]. When the expression of CTHRC1 protein was up-regulated in fibroblasts, it could inhibit the synthesis of type I collagen. CTHRC1 might create suitable extracellular environment for the invasion and migration of tumor cells possibly by reducing the synthesis of the components of extracellular matrix. Therefore, CTHRC1 gene is very promising in cancer diagnosis and the prevention and treatment of recurrence of cancer metastasis.

Therefore, there is urgent need for the precise and specific diagnosis of particular cancer.

### SUMMARY OF THE INVENTION

Accordingly, the objects of the present invention are to provide a diagnostic kit for precise diagnosis of liver cancer, a use of the CTHRC-1 gene in diagnostic kit, and a method for determining the expressing amount thereof in vitro.

In one aspect of the present invention, the use of CTHRC1 nucleic acid sequence or protein in the preparation of an agent for diagnosing liver cancer or a kit comprising the said diagnostic agent is provided.

In one preferred example of the said aspect, the agent for diagnosing liver cancer is an antibody specific against CTHRC1 protein or a nucleic acid probe specific to CTHRC1 protein.

In another aspect of the present invention, a method for detecting liver cancer is provided, which comprises the following steps:

a) administering to an animal the CTHRC1 nucleic acid probe conjugated with radioactive nuclide;

b) detecting the aggregation of said nucleic acid probe in the animal body;

c) the aggregation suggests the presence of liver cancer.

In one preferred embodiment, the radioactive nuclide is α-³²P. In another preferred embodiment, the animal is human.

In another aspect of the present invention, a kit for diagnosing liver cancer is provided. The kit comprises a container that includes anti-CTHRC1 antibodies; and a label to indicate that the kit is used in the diagnosis of liver cancer.

In yet another aspect of the present invention, a kit for diagnosing liver cancer is provided. The kit comprises a container that includes nucleic acid probes specific to CTHRC1; and a label to indicate that the said kit is used in the diagnosis of liver cancer.

Another aspect of the present invention is to provide an in vitro method for detecting specific CTHRC1 protein expression, comprising the steps of:

Reacting an anti-CTHRC1 specific antibody or a nucleic acid probe specific to CTHRC1 with cell sample, with normal liver cell as control;

Comparing the binding amount of said antibody or probe, wherein the amount higher than that of the control indicates that the cell is the cell of liver cancer and the amount lower than or equal to that of the control indicates that the cell is normal.

In one preferred embodiment of the said aspect, the binding amount is determined by detection of the detectable moiety conjugated to the probe or antibody.

In another preferred embodiment of the said aspect, the detectable moiety is selected from chromophore, chemiluminescent moiety, fluorophore or isotope.

### DESCRIPTION OF FIGURES

FIG. 1 shows the expression profile of CTHRC1 in the cells of liver cancer. FIG. 1A shows the RT-PCR results of the cells of liver cancer. FIG. 1B shows a histogram corresponding to FIG. 1A.

FIG. 2 shows the analysis of the CTHRC1 expression profile in 12 liver cancer patients. FIG. 2A is the RT-PCR assay of mRNA expression levels in cancer tissue and normal tissue from liver cancer patient in Hangzhou. FIG. 2B is the RT-PCR assay of the mRNA level of liver cancer patient from Guangxi. In these figures, 1 represents G139; 2, G111; 3, G116; 4, G108; 5, G83; 6, G64; 7, G114; 8, G65.

FIG. 3 shows the assay of the expression of CTHRC1 gene in multiple normal human tissues, in which the numbers are: 1 heart; 2 brain; 3 placenta; 4 lung; 5 liver; 6 bone; 7 kidney; 8 pancreas; 9 spleen; 10 thymus; 11 prostate; 12 testis; 13 ovary; 14 small intestine; 15 colon; 16 peripheral blood lymphocyte.

FIG. 4 shows the effect of CTHRC1 on the migration of MHCC97L cells.

FIG. 5 shows the effect of CTHRC1 on the invasion of MHCC97L cells.

FIG. 6 shows the mRNA sequence of CTHRC1 (gi|34147546|ref|NM_{—}138455.2|) and its amino acid sequence (gi|19923989|ref|NP_{—}612464.1|). These two sequences can be found in Genbank by the above accession numbers.

### MODE OF CARRYING OUT THE INVENTION OR UTILITY MODEL

From the difference between the profiles of gene expression in liver cancer tissue and corresponding liver tissue adjacent to the cancer by genechip technique, the inventor discovered that CTHRC1 had specific high expression in liver cancer tissue.

As used herein, the term “CTHRC1” defines a gene enriched with collagen triple helix structure found in the screening of differential expressed sequences in rat artery and globular injured artery. The CRHRC1 gene used herein includes the complete DNA coding sequence, RNA sequence, mutants, and the functionally active fragments. It should be understood that when encoding the same amino acid, the substitution in the nucleotides of the codon can be acceptable. Furthermore, it should be understood that when replacement of nucleotide results in conservative amino acid replacement, the change of nucleotide is also acceptable.

After obtaining the CTHRC1 nucleic acid fragments, specific probes can be designed based on the nucleotide sequence. The full length nucleotide or the fragment thereof can be obtained by PCR amplification, recombination or artificial synthesis. For PCR amplification, primers can be designed based on the nucleotide sequences disclosed herein, particularly the open reading frame sequence; and cDNA library commercially available or prepared by the common method known in the art can be used as template to amplify the desired sequence. When the sequence is rather long, two or more PCR amplifications generally will be necessary, and then fragments obtained from each amplification will be spiced together in correct order.

Once relevant sequences are obtained, they can be produced in a large scale by recombination method. This usually involves cloning it into vectors, transferring into the cells, and then being isolated from the propagated host cells by common methods.

Additionally, the relevant sequences can be artificially synthesized, particularly when the fragments are relatively short. Often fragments with very long sequences can be obtained by first synthesizing small fragments and then splicing.

At present, the DNA sequence encoding the present protein (or the fragments and derivatives thereof) can be obtained completely by chemical synthesis. Then the DNA sequence can be introduced into various known DNA molecules (or, such as vectors) and cells.

In the present invention, CTHRC1 polynucleotide sequence can be inserted into recombinant expressing vectors. Generally, any plasmids or vectors can be used as long as they can replicate and be stable in the host. One important feature of the expressing vector is that it usually includes replication origin, promoter, marker gene and translation regulation element.

The well known methods by the skilled in the art can be used to construct expressing vectors comprising DNA sequence of CTHRC1 and suitable transcription/translation regulation signals. These methods include in vitro recombinant DNA technique, DNA synthesis technique, in vivo recombination technique, etc. The above-mentioned DNA sequences can be effectively linked to suitable promoters in the expressing vector to direct mRNA synthesis. The transforming vector also comprises ribosome binding site for initiating translation and transcription terminators.

Furthermore, the expressing vectors preferably comprise one or more selective marker genes, to provide the phenotype properties for selecting transformed host cells, for example, dihydro folic acid reductase, neomycin resistance and green fluorescent protein (GFP) in eukaryote cell cultivation, or tetracycline or ampicillin resistance for E. coli.

The vectors comprising the above suitable DNA sequences, promoters or regulation sequences can be used to transform suitable host cells to enable the expression of the protein.

Host cells can be prokaryotic cells, for example bacterial cells; or lower eukaryotic cells, for example yeast cells; or higher eukaryotic cells, for example mammalian cells. Exemplary host cells include E. coli, bacteria cells of Streptomycete, fungal cells such as yeast, plant cells, insect cells, and animal cells, etc.

Transformation of host cells by recombinant DNA can be done by common techniques well known by the skilled in the art. When the host is a prokaryotic organism, for example E. coli, competent cells that absorbs DNA can be obtained in the late phase of exponential growth period, then the cells are processed with CaCl₂, the steps of which are known in the art. Another method is to use MgCl₂. If necessary, the transformation can also be carried out by electroporation. When the host is eukaryotic organism, the following DNA transfection technologies can be chosen: calcium phosphate co-precipitation method, common mechanical method such as microinjection, electroporation, and liposome packaging, etc.

The transformant obtained can be cultivated with common method to express the polypeptide encoded by the present gene. According to the host cell used, the medium in the culture can be selected from all kinds of common mediums. Cultivation is done under the conditions suitable for the host cell growth. When the host cells grow into suitable cell density, suitable method is performed (for example temperature switching or chemical induction) to induce the chosen promoter, and the cells are cultivated for another period of time.

In the above methods, the recombinant polypeptides can be expressed in the cell, on the cell membrane, or be secreted outside. If necessary, various isolation methods can be used to isolate and purify the recombinant proteins based on their physical, chemical or other characters. These methods are well known by the skilled in the art. The examples of the methods include, but not limit to, common renaturation process, process with protein precipitator (salt out), centrifugation, osmotic breaking the bacteria, ultraprocess, ultracentrifuge, molecular sieve chromatography (gel filtration), adsorption chromatography, ion-exchange chromatography, high performance liquid phase chromatography (HPLC) and other liquid phase chromatography technologies, and the combination thereof.

After obtaining the nucleic acid sequence, specific nucleotide probes can be designed based on the nucleic acid sequence. The probe designing methods are common in the art, see Sambrook et al, Molecular Cloning, A laboratory manual, New York: Cold Spring Harbor Laboratory Press, 1989. Exemplary method for detection of the presence of DKK-1 protein or nucleic acid in the biological sample includes obtaining the biological sample of the subject, contacting the biological sample with the labeled nucleic acid probe that can hybridize with DKK-1 mRNA or genomic DNA. The nucleic acid probe can be, for example, human nucleic acid or a part of it, and, for example comprises at least 15, 30, 50, or 100 nucleotides and can sufficiently hybridize with DKK-1 mRNA or genomic DNA under stringent conditions. The other probes used in the present diagnostic assay are mentioned herein.

Nucleic acid probes contact with the amplified sequences which are labeled. The probes are preferably linked to a chromophore, but it also can be radioactively labeled. In another embodiment, the probes are linked to a binding partner, for example antibody or biotin, or another binding partner with detectable domain.

In the traditional methods, the detection can be done by Southern blotting and hybridization with labeled probes. The technology of Southern blotting is well known by the skilled in the art (See Sambrook et. al., 1989). Common detections also include biochips, fluorescence imaging, flow cytometry, etc.

In another aspect, the present invention further comprises polyclonal antibodies and monoclonal antibodies specific against polypeptides encoded by CTHRC1 DNA or the fragments thereof, particularly monoclonal antibodies. Herein, “specificity” or similar terms means that the antibodies can bind to CTHRC1 gene product or the fragment. Preferably it indicates the antibodies that can bind to the DKK-1 gene product or fragment, but unable to recognize or bind to other irrelevant antigen molecules. The present antibodies can be prepared by various technologies known by the skilled in the art.

The present invention not only includes complete monoclonal antibodies or polyclonal antibodies, but also the antibody fragments with immunological activities, for example Fab′ or (Fab)₂ fragments, antibody heavy chains, antibody light chains, single chain Fv molecules engineered by genetic engineering, or chimeric antibodies.

The antibodies against CTHRC1 protein can be used in immunohistochemistry technologies to detect CTHRC1 protein in the biopsy sample, or as a specific therapeutic agent to prevent liver cancer migration and invasion.

Direct detection of CTHRC1 in blood or urine sample can be used as an observation index for tumor assistant diagnosis and prognosis, and also as a basis for early diagnosis of tumor.

Antibodies can be detected by ELISA, Western blotting, or they can be conjugated with detecting moieties, thereby being detected by methods such as chemiluminescence and isotopic tracing.

The present invention also includes kits to perform any methods mentioned herein. In a non-limiting example, the kit will contain one or more reagents in suitable containers. The kit can also include reagents and labels for RNA isolation and RNA purification in the amplified cells.

The components of the kit can be packed in aqueous medium or in lyophilized form. The suitable containers in the kit normally at least include vial, tube, flask, pet, syringe or other containers, which may contain one component, and preferably that it can be suitably divided equally. When there are more than one component in the kit, the kit will normally contain a second, third or other additional containers to deposit the additional components separately. However, components of different combinations can be kept in one vial. The present kit often also includes a container to hold reactants, and is sealed for commercial distribution. The containers can include die cast or blow molded plastic containers, wherein the necessary vials are kept.

The following part will further explain the present invention in combination with particular examples. It should be understood that these examples are only to illustrate the invention without intention of limiting it. The experimental methods in the following examples without special noticing conditions will accord to common conditions, for example those mentioned in Sambrook et al, Molecular Cloning, A laboratory manual, New York: Cold Spring Harbor Laboratory Press, 1989, or as suggested by the manufacturer.

### EXAMPLE 1

### Northern Blotting Assay

1.1 Main Reagents.

Trizol was purchased from Invitrogen, Hybond-N+ film from Amersham, normal human multi-tissue Northern film (MTN blot) and ExpressHyb hybridization solution from Clontech, NEBlot kit from NEB, eukaryotic expressing vector pCMV-3Tag from Stratagene, restriction endonuclease and T4 ligase from Promega, real time PCR kit from American ABI. Taqman-MGB probes and primers for CTHRC1 and housekeeper gene β-actin were designed and synthesized by ABI. Kit for extracting blood/cell/tissue genomic DNA was purchased from Tianwei Time, Beijing. Primers were designed by Primer 3 software. Primers were synthesized by Shanghai shenggong biotechnology service, LLC. Hyclone super fetal calf serum was purchased from Hyclone. High-sugar DMEM was purchased from Gibco. Liposome Lipofectamine™ Reagent was purchased from Invitrogen. Transwell cell with inner diameter of 6.5 mm and pore size of 8.0 mm was purchased from Corning. Matrigel™ was purchased from Corning. The other reagents were all domestically analytical pure.

1.2 Collection of Sample of Cancer Tissue, Liver Tissue Adjacent to the Cancer, and Liver Cancer Cell Strains from Patients with Liver Cancer

The cancer tissue and liver tissue adjacent to the cancer from the liver cancer patients were obtained from Guangxi medical college and First hospital of Zhejiang University medical school. The cell strain MHCC97L of liver cancer with low migration and the cell strain HCCLM3 of liver cancer with high migration were provided by Liver Cancer Institution, Zhongshan Hospital of Fudan University. Other strains were kept by the inventor's lab (HepG2 cell, Hep3B cell, MHCC97L cell, HCCLM3 cell, HuH7 cell). The tissue samples after surgery are immediately frozen in liquid nitrogen, and then stored in −80° C. ultralow temperature refrigerator.

1.3 Cell Culture

5 strains HepG2, Hep3B MHCC97L, HCCLM3, HuH7 were all cultivated in DMEM medium with 10% fetal calf serum, with addition of 100 U/ml penicillin and 100 μg/ml streptomycin, and incubated at 37° C. in 5% CO₂.

1.4 RT-PCR Analysis and Northern Blotting Assay

CTHRC1 expression profile was detected with RT-PCR method in the liver cancer cell strains. Total RNA of 8 liver cancer cell strains were extracted with Trizol, in which 5 μl of each total RNA were used for reverse transcription under the following condition: 65° C. 5 min, 50° C. 50 min, 85° C. 5 min, 37° C. 20 min. 1 μl of the product was diluted 5 times, then used as template for PCR amplification. Amplification product was obtained before the reaction reached platform phase and then detected by electrophoresis. β-actin was used as inner control. The primer sequences were as follows: Forward primer 5′-TGGATGGAATTCAGTTTCTCGCATCA-3′Reverse primer 5′-GCTTCAATCAAAAGTGGTTTCAA-3′. PCR reaction conditions were as follows: pre-denaturing at 94° C. for 2 min; denaturing at 94° C. for 30 s, annealing at 58° C. for 30 s, extending at 72° C. for 45 s, totally 30 cycles; and extending at 72° C. for 5 min.

Real-time PCR analysis was done for the carcinoma tissue and liver tissue adjacent to cancer from 8 liver cancer patients (G139, G111, G116, G108, G83, G64, G114, G65). The RNA extraction and reverse transcription processes were the same as those of RT-PCR. The reaction conditions of real time PCR were 50° C. 2 min, 95° C. 10 min; 95° C. 15 s, 60° C. 1 min for 40 cycles. PCR reaction was performed on ABI7300 device. Fluorescent value was real-time monitored in the extension phase of each cycle. Data analysis was done automatically by ABI3700 system software.

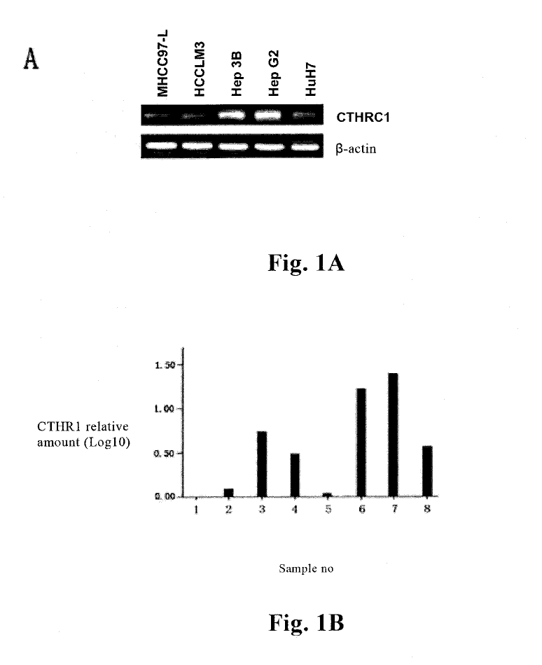

The experiment results were shown in FIG. 1. FIG. 1 showed the expression profile of CTHRC1 in the liver cancer strains. FIG. 1A showed the result of fluorescent quantitative PCR. FIG. 1B showed the corresponding result from RT-PCR. The result showed that in 5 liver cancer cell strains, CTHRC1 was highly expressed in HepG2, Hep3B, MHCC97L, HCCLM3, and HuH7.

The above 5 human liver cancer cell strains were assayed by Northern blotting. Total RNA of human liver cancer cell strains were extracted with Trizol. Each sample of 10 mg total RNA were subjected to electrophoresis on 1% formaldehyde denatured gel, and then transferred to a Hybond-N+ film. 837 bp CTHRC1 gene probe (the 58 bp to 894 bp of the GenBank Accession Number NM_{—}138455.2 sequence) was labeled with [α-³²P]dCTP. NEBlot kit was used for Nuclide labeling. Then the hybridization reaction was done by using ExpressHyb hybridizing solution. The result of Northern blotting (with β-actin as control) showed that CTHRC1 was specifically expressed in all these 5 liver cancer cells.

### EXAMPLE 2

### The Expression Profile of CTHRC1 in the Tissue of Patients with Liver Cancer

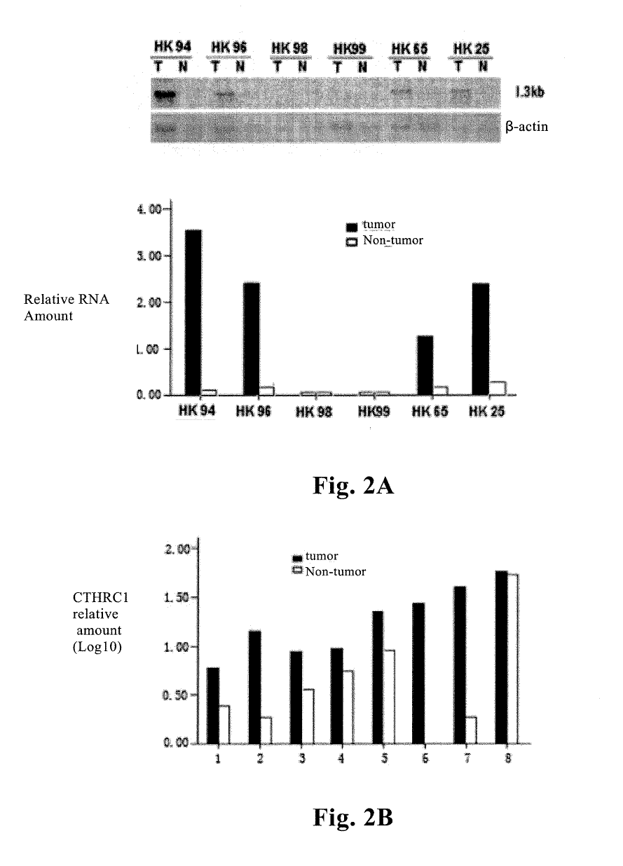

14 pairs of liver cancer and the corresponding peritumoral tissue are tested, with β-actin as inner control, wherein the HK group were the patients from Hangzhou and the G group were the patients from Guangxi, wherein 6 pairs of liver cancer and corresponding peritumoral tissue from Hangzhou patients were tested by Northern blotting. 8 pairs of liver cancer and corresponding peritumoral tissue from the Guangxi patients were tested by fluorescent quantitative PCR assay. The results showed that in 11 pairs of samples the expression of CTHRC1 were higher than those in peritumoral tissues (P<0.01), and the rest 3 pairs did not have statistical difference (FIG. 2). The results indicated that in most patients with liver cancer, CTHRC1 was specifically expressed in liver cancer tissue.

### EXAMPLE 3

### Northern Blotting Assay for CTHRC1 Expression in Normal Human Tissue

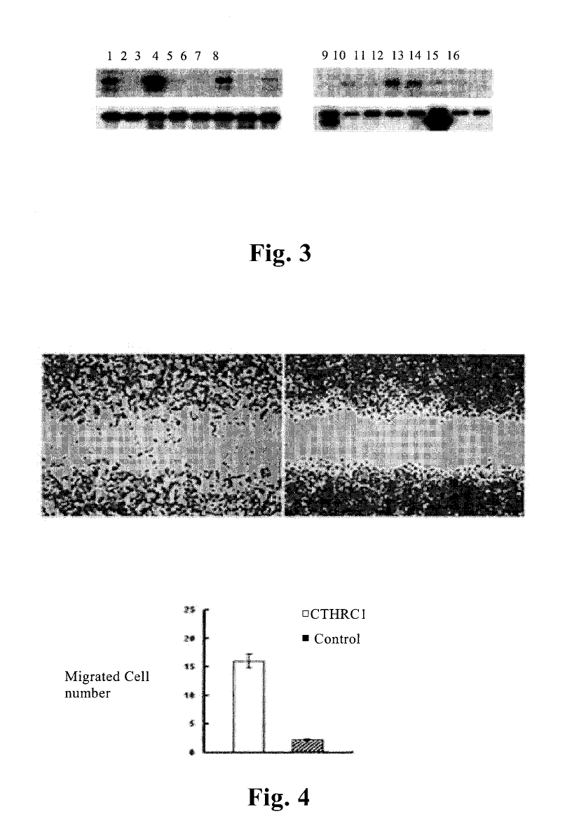

Northern blotting was used to analyze the expression profile of 16 normal human tissues. Gray-scale was performed and then the results was plotted as a statistic graphic (FIG. 3). The results showed that in most normal human tissues, CTHRC1 did not specifically express.

### EXAMPLE 4

### Injure Repair Assay

4.1 The Construction of pCMV-3Tag-CTHRC1 eukaryotic Expressing Vector

The complete coding region sequence of CTHRC1 gene was cloned from human placenta cDNA. The forward primer was 5′-AAGGAAAAAAGCGGCCGCGCCACCATGCGACCCCAGGGC-3′, and the reverse primer was 5′-CCGCTCGAGATTTTGGTAGTTCTTC-3′. PCR reaction conditions were as follows: pre-denaturing at 94° C. for 2 min; denaturing at 94° C. for 30 s, annealing at 52° C. for 30 s, extending at 72° C. for 1 min, totally 35 cycles; extending at 72° C. for 5 min. PCR product was confirmed by 1% agar gel electrophoresis, and target fragments of about 765 bp was purified and recovered. The PCR amplified fragments of pCMV-3Tag plasmid and CTHRC1 were digested with NotI and XhoI, and the digested pCMV-3Tag vectors and the CTHRC1 fragments were purified and recovered. Target fragment and the linear null vector pCMV-3Tag-9 were spliced by T4 DNA ligase with molar ratio of 3:1, and then reacted overnight at 16° C. The ligated product was used to transform TOP10 competent bacteria, and the transformed bacteria broth was plated on LB plate with ampicillin, and incubated for 12 h. Positive clones were picked to perform minor amplification and vector extraction. Positive bacterial clone was identified by ApaI digestion. Vectors were sequenced in Shanghai Ding'an biotech LLC, and the result confirmed the successful construction of the pCMV-3Tag-CTHRC1 eukaryotic expressing vector.

4.2 Scratch Wound Assay

MHCC97L cells in good growth condition were inoculated in 12-well plate at 3×10⁵cells/well. Cells were incubated in DMEM medium with 10% FBS at 37° C., 5% CO₂. Transfection was done after entire confluence. 2 ml of LIPOFECTAMINE2000 (1 mg/ml) and 0.5 mg of pCMV-3Tag-CTHRC1 plasmid DNA (or null vector pCMV-3Tag) were diluted by DMEM to 100 ml respectively and then mixed together, and placed at room temperature for 20 min. Cells were washed by serum-free DMEM twice. The mixture of DNA and liposome were added to 1 ml by serum-free DMEM, and then used to transfect the cells. After 5 h, the medium was changed by DMEM with 1 ml 10% FBS. After 24 h, each well was scratched by 200 μl tip, and rinsed 3 times with serum-free DMEM medium. DMEM medium with 2% FBS was added and incubation continued for 24 h. At the end of the experiment, cells were fixed in the well and dyed with crystal violet, counted under microscope and photos were taken. Each experimental and control group have 3 parallel samples, and each sample were observed under 2 visual fields (object lens, ×10). The numbers of migrated cells were: 16.0+2.65, 3.33+1.16 (FIG. 4), respectively. The differences were statistically evident (P<0.05).

### EXAMPLE 5

### In vitro Invasion Assay

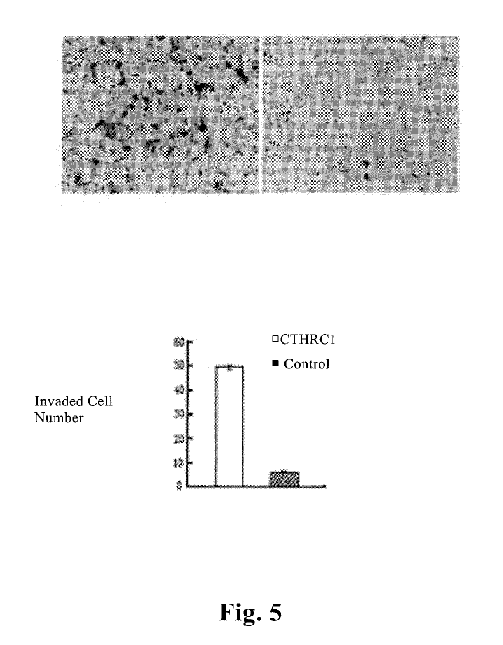

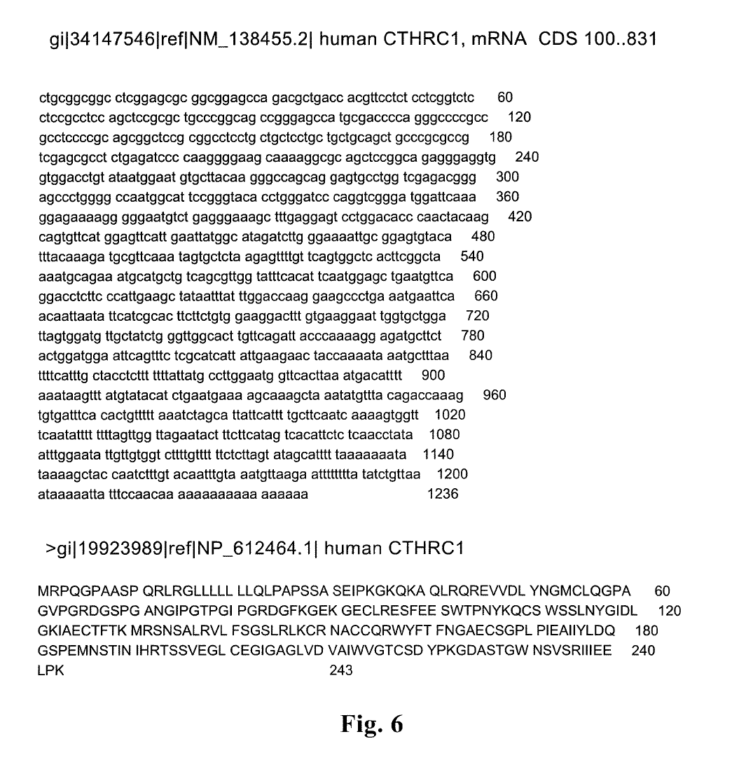

MHCC97L cells in good growth condition were inoculated in 12-well plate at 3×10⁵ cells/well. Cells were incubated in DMEM medium with 10% FBS at 37° C., 5% CO₂. Transfection was done after entire confluence. 2 ml of LIPOFECTAMINE2000 (1 mg/ml) and 0.5 mg of pCMV-3Tag-CTHRC1 plasmid DNA (or null vector pCMV-3Tag) were diluted by DMEM to 100 ml respectively and then mixed together, and placed at room temperature for 20 min. Cells were washed by serum-free DMEM twice. The mixture of DNA and liposome were added with serum-free DMEM to a total volume of 1 ml, and then used to transfect the cells. After 5 h, the medium was changed by DMEM with 1 ml 10% FBS. After 24 h, each well was scratched by 200 μl tip, and rinsed 3 times with serum-free DMEM medium. DMEM medium with 2% FBS was added and incubation continued for 24 h. At the end of the experiment, cells were fixed in the well and dyed with crystal violet, counted under microscope and photos were taken. Each experimental and control group have 3 parallel samples, and each sample were observed in the central visual field (object lens, ×10), totally 3 visual fields. The numbers of invasion cells were: 49.3+6.66, 8+1.87. FIG. 5A showed the visual field pictures of one group of experimental and control samples. FIG. 5B was a histogram of the mean value of 3 samples. The differences were statistically evident (P<0.01). The experimental result showed that the invasion ability of MHCC97-L CTHRC1 (experimental group) cells was evidently higher than that of the control group (P<0.05), which proved that over-expressing CTHRC1 could improve the invasion ability of MHCC97-L of the liver cancer cell.

Summing up, the CTHRC1 gene is closely related to liver cancer, and is evidently specific in the liver cancer tissue and cells. Furthermore, it was noticed that after transfected by CTHRC1, the invasion and migration abilities of the liver cancer cell strain MHCC97L with low migration ability were greatly improved, which indicates that the said gene is closely related to the migration and invasion of liver cancer, and can be used as a target for preventing and treating invasive and metastatic liver cancer.

All references mentioned in this application are herein incorporated by reference into the present application to the same extent as if each was specifically and individually indicated to be incorporated herein by reference. Additionally, it will be understood that in light of the above disclosure of the present invention, those skilled in the art can make various changes and modifications, all of which fall in the scope of the claims of the present invention.

### REFERENCES

[1] PARKIN D M. Global cancer statistics in the year 2000. Lancet Oncol. 2001 Sep.; 2(9):533-543.

[2] PYAGAY P, HEROULT M, WANG Q, et. al., Collagen triple helix repeat containing 1, a novel secreted protein in injured and diseased arteries, inhibits collagen expression and promotes cell migration. Circ Res. 2005 Feb 4; 96(2):261-268.

[3] LI Y, TANG Z Y, YE S L, et. al., Establishment of cell clones with different metastatic potential from the metastatic hepatocellular carcinoma cell line MHCC97. World J Gastroenterol, 2001 Dec; 7(6): 777-778.

[4] TANG L, DAI D L, SU M, et. al., Aberrant expression of collagen triple helix repeat containing 1 in human solid cancers. Clin Cancer Res 2006 Jun 15; 12(12):3716-3722.

[5] DVORAK H F. Tumors: wounds that do not heal. Similarities between tumor stroma generation and wound healing. N Engl J Med. 1986 Dec 25; 315(26):1650-1659.

[6] BEACHY P A, KARHADKAR S S, BERMAN D M. Tissue repair and stem cell renewal in carcinogenesis. Nature. 2004 Nov 18; 432(7015):324-331.

[7] COUSSENS L M, WERB Z. Inflammation and cancer. Nature 2002 Dec 19-26; 420(6917): 860-867.

[8] ALLINEN M, BEROUKHIM R, CAI L, et. al., Molecular characterization of the tumor microenvironment in breast cancer. Cancer Cell. 2004 Jul; 6(1):17-32.

[9] WEST R B, NUYTEN D S, SUBRAMANIAN S, et. al.,. Determination of stromal signatures in breast carcinoma. PLoS Biol 2005 Jun; 3(6):e187.

[10] MICKE P, OSTMAN A. Tumour-stroma interaction: cancer-associated fibroblasts as novel targets in anti-cancer therapy? Lung Cancer 2004 Aug, 45 Suppl 2: S163-175.

[11] KATAOKA H, TANAKA H, NAGAIKE K, et. al., Role of cancer cell-stroma interaction in invasive growth of cancer cells. Hum Cell 2003 Mar, 16(1): 1-14.

[12] BHOWMICK N A, NEILSON E G, MOSES H L. Stromal fibroblasts in cancer initiation and progression. Nature. 2004 Nov 18; 432(7015):332-7. Review.

1. Use of CTHRC1 nucleic acid sequence or protein in the preparation of agent for diagnosing liver cancer or kit comprising the said agent.

2. The use according to claim 1, wherein the agent for diagnosing liver cancer is an antibody specific against CTHRC1 protein or a nucleic acid probe specific to the CTHRC1 protein.

3. A method for detecting liver cancer, comprising the steps of:
a) Administering a CTHRC1 nucleic acid probe conjugated to a radioactive nuclide to an animal;
b) detecting the aggregation of said nucleic acid probe in the animal body;
c) the aggregation suggests the presence of liver cancer.

4. The method according to claim 3, wherein the radioactive nuclide is α-³²P.

5. The method according to claim 3, wherein the said animal is human.

6. A kit for diagnosing liver cancer comprising a container that includes anti-CTHRC1 antibodies; and a label to indicate that the said kit is used in the diagnosis of liver cancer.

7. A kit for diagnosing liver cancer comprising a container that includes nucleic acid probes specific to CTHRC1; and a label to indicate that the said kit is used in the diagnosis of liver cancer.

8. An in vitro method for detecting specific CTHRC1 protein expression, comprising the steps of:
Reacting anti-CTHRC1 specific antibodies or nucleic acid probe specific to CTHRC1 with cell sample, with normal liver cell as control;
Comparing the binding amount of antibodies or probes, wherein the amount higher than that of control indicates that the cell is the liver cancer cell and the amount lower than or equal to that of the control indicates that the cell is normal.

9. The method according to claim 8, wherein the binding amount is determined by detection of the detectable moiety conjugated to the probe or antibody.

10. The method according to claim 9, wherein the detectable moiety is selected from the group consisting of chromophore, chemiluminescent moiety, fluorophore or isotope.
